(19) United States
(12) Patent Application Publication
(10) Pub. No.: US 2026/0259224 A1
(43) Pub. Date: Sep. 3, 2026

(54) BIOMARKER OF SKIN AGING, METHOD FOR DETERMINING SKIN AGING, AND METHOD FOR SCREENING SUBSTANCE FOR IMPROVING SKIN AGING

(71) Applicant: ROHTO PHARMACEUTICAL CO., LTD., Osaka-shi, Osaka (JP)

(72) Inventors: Florence FLORENCE, Osaka-shi (JP); Masatoshi HAGA, Osaka-shi (JP); Yasunari SATO, Osaka-shi (JP)

(73) Assignee: ROHTO PHARMACEUTICAL CO., LTD., Osaka-shi, Osaka (JP)

(21) Appl. No.: 18/875,346

(22) PCT Filed: Jun. 19, 2023
(86) PCT No.: PCT/JP2023/022650

§ 371 Date: Dec. 16, 2024

Related U.S. Application Data
(60) Provisional application No. 63/353,070, filed on Jun. 17, 2022.
(60) Provisional application No. 63/403,991, filed on Sep. 6, 2022.

(51) Int. Cl. G01N 33/68

(52) U.S. Cl. CPC G01N 33/6887; G01N 2333/4722; G01N 2333/59; G01N 2333/78; G01N 2500/04; G01N 2800/20; G01N 2800/7042

(57) ABSTRACT

An object of the present invention is to provide a biomarker for use in determining skin aging, the biomarker containing a protein or a fragment thereof. Another object of the present invention is to provide a method for determining skin aging in a subject, the method being characterized by quantifying a protein or a fragment thereof in a blood sample from the subject. Yet another object of the present invention is to provide a method for screening a substance for improving skin aging, the method being characterized by examining a change in expression level or concentration of a protein or a fragment thereof in a blood sample from a subject before and after ingestion of a test substance by the subject.

The present invention is directed to a biomarker for use in determining skin aging, the biomarker containing a protein or a fragment thereof, the protein being selected from the group consisting of osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).

The present patent application claims priority to U.S. Provisional Application No. 63/353,070 filed on Jun. 17, 2022 and U.S. Provisional Application No. 63/403,991 filed on Sep. 6, 2022, the contents of which were published in Florence. et al. (2023). IFSCC Magazine, 26 (1), 7-16. and are incorporated herein by reference in their entirety.

### TECHNICAL FIELD

The present invention relates to a biomarker of skin aging, a method for determining skin aging, and a method for screening a substance for improving skin aging.

### BACKGROUND ART

Skin aging is a multifaceted process affected by various intrinsic factors (e.g., cell aging) and extrinsic factors (e.g., such as ultraviolet light and irritants) (Non-Patent Literature 1).

Regarding parabiosis between a young mouse and an old mouse, such as those sharing a circulatory system, it has been reported that a factor present in young serum exerts a regenerative effect on tissues and organs of the old mouse while a degenerative effect by a factor derived from old serum is observed in organs of the young mouse (Non-Patent Literature 2). Based on this, a hypothesis has been proposed that molecular changes in blood associated with advancing age may contribute to aging of living bodies (Non-Patent Literature 3). However, studies on the effects of changes of proteins in blood on the skin cannot be said to be sufficient yet. In particular, effects due to changes of proteins in blood as intrinsic factors of skin aging are not sufficiently known.

### CITATION LIST

### Non-Patent Literature

Non-Patent Literature 1: Zhang, S., & Duan, E. (2018). Cell transplantation, 27(5), 729-738.
Non-Patent Literature 2: Conboy, I. et al. (2005). Nature, 433(7027), 760-764.
Non-Patent Literature 3: Lehallier, B. et al. (2019). Nature medicine, 25(12), 1843-1850

### SUMMARY OF INVENTION

### Technical Problem

An object of the present invention is to provide a biomarker for use in determining skin aging, the biomarker containing a protein or a fragment thereof. Another object of the present invention is to provide a method for determining skin aging in a subject, the method being characterized by quantifying a protein or a fragment thereof in a blood sample from the subject. Yet another object of the present invention is to provide a method for screening a substance for improving skin aging, the method being characterized by examining a change in expression level or concentration of a protein or a fragment thereof in a blood sample from a subject before and after ingestion of a test substance by the subject.

### Solution to Problem

As a result of diligent research to solve the above problems, the present inventors have first found a blood protein that increases in a gender-specific manner with advancing age, and have completed the present invention. Also, the present inventors have first found that such a protein inhibits collagen fiber formation in fibroblasts, inhibits collagen precursor secretion, and causes abnormalities in the fibril structure of the extracellular matrix. In addition, the present inventors have revealed that such a protein inhibits the barrier function in the epidermis and inhibits the expression of a type I collagen precursor in the dermis. Based on these findings, the present inventors have developed a biomarker for use in determining skin aging, a method for determining skin aging, and a method for screening a substance for improving skin aging.

Accordingly, the gist of the present invention is as follows.

[1] A biomarker for use in determining skin aging, the biomarker containing a protein or a fragment thereof, the protein being selected from the group consisting of osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).

[2] The biomarker according to [1], wherein the protein or a fragment thereof is contained in a blood sample from a subject.

[3] The biomarker according to [2], wherein an increase in expression or concentration of the protein or a fragment thereof in the blood sample from the subject indicates progression of skin aging in the subject.

[4] The biomarker according to [2], wherein a decrease in expression or concentration of the protein or a fragment thereof in the blood sample from the subject indicates improvement of skin aging in the subject.

[5] A method for determining skin aging in a subject, the method being characterized by quantifying at least one protein or a fragment thereof in a blood sample from the subject, the protein being selected from the group consisting of osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).

[6] A method for screening a substance for improving skin aging, the method being characterized by examining a change in expression level or concentration of at least one protein or a fragment thereof in a blood sample from a subject before and after ingestion of a test substance by the subject, the protein being selected from the group consisting of osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).

### Advantageous Effects of Invention

The biomarker of skin aging of the present invention is a protein that is increased by skin aging and thus is used as a physiological indicator indicating progression of aging. The method for determining skin aging of the present invention quantifies a protein that increases in the skin in which aging progresses, that is, the biomarker of skin aging of the present invention, to determine skin aging. The method for screening a substance for improving skin aging of the present invention examines a change in concentration of the biomarker protein of skin aging of the present invention before and after ingestion of a test substance and can find a substance for improving skin aging. A substance for improving skin aging to be found by the method of the present invention can control symptoms related to skin aging and thus can be expected to be effective for symptoms caused by progression of skin aging.

### BRIEF DESCRIPTION OF DRAWINGS

FIG. 1 is a series of graphs showing z-score trajectories for eight proteins over time in females and males.

FIG. 2A is a series of immunofluorescence images of type I collagen fibers of NHDF cells among evaluation results on collagen production of osteomodulin in Test Example 2.

FIG. 2B is a graph showing results of collagen production quantity among evaluation results on collagen production of osteomodulin in Test Example 2.

FIG. 2C is a series of scanning electron microscope images of extracellular matrices of NHDFs among evaluation results on collagen production of osteomodulin in Test Example 2.

FIG. 2D is a series of graphs showing results of quantification and distribution of collagen fiber diameters among evaluation results on collagen production of osteomodulin in Test Example 2.

FIG. 3 is a series of graphs showing results of gene expression in Test Example 3 (A: OCLN, B: OLDNI, C: FLG, D: LOR, and E: KRT1).

FIG. 4A is a series of images showing H&E staining of human skin equivalents (HSEs) among results of histological analysis of HSEs in Test Example 4.

FIG. 4B is a graph showing measurement results of transepidermal water loss (TEWL) among results of histological analysis of human skin equivalents (HSEs) in Test Example 4.

FIG. 4C is a series of images showing immunostaining of KRT1/KRT14 among results of histological analysis of human skin equivalents (HSEs) in Test Example 4.

FIG. 4D is a series of images showing immunostaining of FLG/CLDN1 among results of histological analysis of human skin equivalents (HSEs) in Test Example 4.

FIG. 4E is a series of images showing immunostaining of Ki67 among results of histological analysis of human skin equivalents (HSEs) in Test Example 4.

FIG. 4F is a series of graphs showing results of gene expression levels of KRT1, FLG, LOR, OCLN, and CLDN1 in epidermis of human skin equivalents (HSEs) among results of histological analysis of HSEs in Test Example 4.

FIG. 4G is a graph showing results of gene expression levels of COL1A1 in dermis of human skin equivalents (HSEs) among results of histological analysis of the HSEs in Test Example 4.

FIG. 5A is a series of immunostaining fluorescence images of type I collagen fibers of NHDF cells among results of evaluation on collagen production of CHAD and LRRC15 in Test Example 5.

FIG. 5B is a graph showing results of collagen production quantity among results of evaluation on collagen production of CHAD and LRRC15 in Test Example 5.

### DESCRIPTION OF EMBODIMENTS

Embodiments of the present invention will be described in detail below. However, the present invention is not limited to the embodiments below. The terms used in the present specification are interpreted in meanings commonly used in the art unless otherwise mentioned.

### [Biomarker]

A biomarker according to the present embodiment is an aging biomarker, which means a factor that varies with advancing age, and is capable of determining and predicting aging, in particular, an aging state, such as degree of progression of skin aging. In addition, the determination and prediction of aging by the biomarker according to the present embodiment can determine and predict improvement and deterioration of aging. The improvement of aging means suppression of aging conditions, inhibition of progression of aging, and promotion of reversal of aging (rejuvenation). As used herein, the aging means functional decline that emerges in individuals, organs, tissues, cells, or the like after the growth period. Skin aging means, in a narrow sense, functional decline of the skin that emerges with advancing age and means, in a broad sense, functional decline of the skin that occurs under the effect of various intrinsic factors (e.g., cell aging) and extrinsic factors (e.g., such as ultraviolet light and irritants). The biomarker according to the present embodiment can be an indicator of physiological aging conditions or pathological aging conditions, particularly physiological aging conditions or pathological aging conditions of the skin. Physiological aging means a physiological functional decline that inevitably progresses with advancing age, and pathological aging means a functional decline, such as those leading to morbidity.

The biomarker according to the present embodiment is a biomarker for use in determining skin aging and contains a protein or a fragment thereof, the protein being selected from the group consisting of osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).

The above-described proteins or fragments thereof are a group of proteins that increase in blood with advancing age, particularly in females, and act on skin cells in the direction of promoting aging. Each protein of the biomarker is a protein known in the art and has characteristics shown in Tables 1-1 and 1-2.

TABLE 1-1

| | Gene | UniProt | Size | |
|---|---|---|---|---|
| Protein Name | (Entrez Gene ID) | ID | (kDa) | GO Biological Processes |
| Collagen type XI | COL11A2 | P13942 | 172 | Cartilage development, collagen fibril organization |
| alpha 2 chain | | | | |
| Chondroadherin | CHAD | Q15335 | 40 | Bone development, negative regulation of bone |
| | | | | trabecula formation |
| Osteomodulin | OMD | Q99983 | 49 | Regulation of bone mineralization, cell adhesion |
| Leucine-rich repeat- | LRRC15 | Q8TF66 | 64 | Negative regulation of protein localization to plasma |
| containing protein 15 | | | | membrane, positive regulation of cell migration, |
| | | | | receptor-mediated virion attachment to host cell |
| Glycoprotein | CGA.CGB3.CGB7 | P01215 | ~27 | Developmental growth, follicle-stimulating hormone |
| hormones alpha | | (CGA) | | secretion, follicle-stimulating hormone signaling |
| chain/ | | P0DN87 | | pathway, G protein-coupled receptor signaling |
| choriogonadotropin | | (CGB7) | | pathway, hormone-mediated signaling pathway, |
| subunit beta 3/ | | | | luteinizing hormone secretion, negative regulation of |
| chorlogonadotropin | | | | organ growth, positive regulation of cell migration |
| subunit beta 7 | | | | positive regulation of cell population proliferation, |
| | | | | positive regulation of steroid biosynthetic process, |
| | | | | positive regulation of transcription by RNA |
| | | | | polymerase II, regulation of signaling receptor activity, |
| | | | | thyroid gland development, thyroid hormone |
| | | | | generation |

| Protein Name | GO Molecular Function | GO Cellular Component |
|---|---|---|
| Collagen type XI | Extracellular matrix structural constituent, | Collagen trimer, collagen type XI trimer2, |
| alpha 2 chain | extracellular matrix structural constituent | collagen-containing, endoplasmic |
| | conferring tensile strength, metal ion binding, | reticulum lumen, extracellular matrix, |
| | protein-macromolecule adaptor activity | extracellular region, extracellular space |
| Chondroadherin | — | Extracellular matrix, extracellular space |
| Osteomodulin | — | Collagen-containing extracellular matrix, |
| | | extracellular region, extracellular space, |
| | | Golgi lumen, lysosomal lumen, |
| | | extracellular exosome |
| Leucine-rich repeat- | Collagen binding, fibronectin binding, laminin | Extracellular exosome, extracellular |
| containing protein 15 | binding | matrix, extracellular space, integral |
| | | component of membrane |
| Glycoprotein | Follicle-stimulating hormone activity, hormone | Extracellular region, extracellular space, |
| hormones alpha | activity | follicle-stimulating hormone complex, |
| chain/ | | Golgi lumen, pituitary gonadotropin |
| choriogonadotropin | | |
| subunit beta 3/ | | |
| chorlogonadotropin | | |
| subunit beta 7 | | |

TABLE 1-2

| | Gene | UniProt | Size | |
|---|---|---|---|---|
| Protein Name | (Entrez Gene ID) | ID | (kDa) | GO Biological Processes |
| Lutropin subunit beta | LHB | Q8WXL0 | 14 | — |
| Glycoprotein | CGA.LHB | Q8WXL0 | ~27 | Similar with CGA.CGB and LHB |
| hormones alpha | | (LHB) | | |
| chain/lutropin subunit | | | | |
| beta | | | | |
| Glycoprotein | CGA.FSHB | P01225 | ~27 | female gamete generation, female pregnancy, |
| hormones alpha | | (FSHB) | | follicle-stimulating hormone signaling pathway, G |
| chain/follitropin | | | | protein-coupled receptor signaling pathway, positive |
| subunit beta | | | | regulation of bone resorption, positive regulation of |
| | | | | cell migration, positive regulation of cell population |
| | | | | proliferation, positive regulation of gene expression, |
| | | | | positive regulation of steroid biosynthetic process, |
| | | | | positive regulation of transcription by RNA |
| | | | | polymerase II, progesterone biosynthetic process, |
| | | | | regulation of osteoclast differentiation, regulation of |
| | | | | signaling receptor activity, Sertoli cell proliferation, |
| | | | | spermatogenesis, transforming growth factor beta |
| | | | | receptor signaling pathway |

| Protein Name | GO Molecular Function | GO Cellular Component |
|---|---|---|
| Lutropin subunit beta | Hormone activity | Extracellular region |
| Glycoprotein | Similar with CGA and LHB | Similar with CGA.CGB and LHB |
| hormones alpha | | |
| chain/lutropin subunit | | |
| beta | | |
| Glycoprotein | — | Cytoplasm, extracellular region, |
| hormones alpha | | extracellular space, follicle-stimulating |
| chain/follitropin | | hormone complex, follicle-stimulating |
| subunit beta | | hormone activity |

Gene IDs are obtained from the Entrez Gene resource of the NCBI. The biological process, molecular functions, and cellular component information is obtained from the Gene Ontology (GO) database.

Among the above eight proteins, the protein of the biomarker according to the present embodiment is preferably osteomodulin (OMD), chondroadherin (CHAD), or leucine-rich repeat-containing protein 15 (LRRC15) from the viewpoint of sensitivity for determining skin aging. From the viewpoint of sensitivity for determining skin aging, the size of the protein of the biomarker is preferably 180 kDa or less, more preferably 150 kDa or less, even more preferably 120 kDa or less, still more preferably 80 kDa or less, and particularly preferably 66 kDa or less.

In quantification of the biomarker according to the present embodiment, a body fluid sample is preferably used as a subject sample, and examples include blood, serum, plasma, urine, secretion, interstitial fluid, synovial fluid, cerebrospinal fluid, and lymph fluid. Among these, blood, serum, or plasma is more preferred, and serum or plasma is even more preferred. The protein or a fragment thereof is contained in a subject sample, preferably in a body fluid sample, more preferably in a blood sample, and even more preferably in a serum sample.

The subject is preferably a mammal or a human, more preferably a human, and even more preferably a biologically female human.

In the biomarker according to the present embodiment, an increase in expression or concentration of the protein or a fragment thereof in a subject sample (preferably in a body fluid sample, more preferably in a blood sample, and even more preferably in a serum sample) indicates progression of skin aging in the subject. On the contrary, a decrease in expression or concentration of the protein or a fragment thereof in a subject sample (preferably in a body fluid sample, more preferably in a blood sample, and even more preferably in a serum sample) indicates improvement of skin aging in the subject. The subject sample may be suitably prepared according to the measurement method after collection from the subject.

Any protein detection or quantification method known in the art can be used as a method for measuring the expression level, concentration, and the like of the biomarker according to the present embodiment using the subject sample. Examples of the method include enzyme-linked immunosorbent assay (ELISA), radioimmunoassay (RIA), immunoprecipitation, and Western blotting. Among these, ELISA is simple and preferred.

In one embodiment of ELISA, a sample containing a biomarker protein according to the present embodiment is exposed to a solid support (e.g., a microtiter plate). The biomarker protein in the sample is immobilized on the support and directly detected using an enzyme-conjugated antibody specific to the biomarker protein.

### [Method for Determining Skin Aging]

A method for determining skin aging of the present invention is a method for determining skin aging in a subject, the method being characterized by quantifying at least one protein or a fragment thereof in a blood sample from the subject, the protein being selected from the group consisting of osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).

The method for determining skin aging according to the present embodiment is characterized by including:
(i) measuring the amount or concentration of each of the above-described proteins or a fragment thereof in a body fluid sample from a subject, preferably in a blood sample from the subject and more preferably in a serum sample from the subject; and
(ii) determining skin aging in the subject by comparing a measured value obtained in (i) with a reference value used as a reference for determining skin aging. The reference value may be a body fluid level, preferably a blood level, more preferably a serum level of a healthy individual, an average value in healthy individuals of the age of the subject, a previously measured value of the subject, or an average value in persons having similar conditions to those of the subject. When the measured value obtained in step (i) above is higher than the reference value, it is determined that skin aging progresses more than the standard, or the skin aging has progressed more than before. On the contrary, when the measured value obtained in step (i) above is lower than the reference value, it is determined that skin aging is delayed from the standard, progression of skin aging is slower than the standard, skin aging has been improved than before, progression of skin aging has been inhibited, or reversal of skin aging (rejuvenation) has occurred. According to the determination method of the present embodiment, the progression state of skin aging can be appropriately evaluated by the biomarker. The protein or a fragment thereof used in the method corresponds to the biomarker of the present invention described above, and the description in the section of Biomarker (paragraphs to [0022] to [0022]) can be applied to the specific description.

### [Method for Screening Substance for Improving Skin Aging]

The method for screening a substance for improving skin aging of the present invention is characterized by examining a change in expression level or concentration of at least one protein or a fragment thereof in a blood sample from a subject before and after ingestion of a test substance by the subject, the protein being selected from the group consisting of osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).

The method for screening a substance for improving skin aging according to the present embodiment includes:
(i) measuring the amount or concentration of the protein or a fragment thereof in a blood sample from a subject before ingestion of a test substance;
(ii) allowing the subject to ingest the test substance;
(iii) measuring the amount or concentration of the protein or a fragment thereof (the same as that measured in step (i) above) in a blood sample from the subject after a certain period of time from step (ii) above; and
(iv) comparing measured values of (i) and (iii) above, and determining that the test substance is a substance for improving skin aging when the value of (iii) is lower than that of (i) or the values of (i) and (iii) are the same, or determining that the test substance is not a substance for improving skin aging when the value of (iii) is higher than that of (i). The method of the present invention makes it possible to find a health care material, a pharmaceutical material, a cosmetic material, and/or the like that improves and/or prevents skin aging, inhibits progression of skin aging, and/or promotes the reversal of aging (rejuvenation). The protein or a fragment thereof used in the method corresponds to the biomarker of the present invention described above, and the description in the section of Biomarker (paragraphs [0012] to [0022]) can be applied to the specific description.

### EXAMPLES

The present invention will be specifically described based on test examples below, but the present invention is not limited by these. In addition, blending amounts in the following test examples and the like are represented by mass % unless otherwise specified. For the test conditions, such as culture conditions in each test example, test examples were carried out under the same conditions except for the difference of a substance to be added unless otherwise specified. Furthermore, a control and/or a negative control was prepared for every test to accurately evaluate the effect of the biomarker of the present invention.

Unless otherwise specified, the following cell cultures, materials, and methods were used in test examples below.

### <Cell Cultures and Materials>

Dermal fibroblasts: Neonatal normal human dermal fibroblasts (NHDFs (NB)), lot number 05882 (Kurabo, Osaka, Japan) were used and cultured in Dulbecco's modified Eagle's medium (DMEM; Gibco, USA) supplemented with a 10% fetal bovine serum, a 1% antibiotic, and an antifungal agent.
Cultured human keratinocytes: Neonatal normal human epidermal keratinocytes (NHEK (NB)), lot number 7609 (Kurabo, Osaka, Japan) were used and cultured in Humedia-KG2 medium (Kurabo, Japan).
Cell culture conditions: Cells were cultured at 37° C. in a 5% CO₂ incubator.
Recombinant human OMD (ab27632) was obtained from Abcam (Cambridge, UK).
A pro-collagen 1 ELISA kit (Human Pro-Collagen I alpha 1 DuoSet ELISA, DY6220-05) was purchased from R&D Systems (Minneapolis, Minnesota, USA).

### Methods

### Method for Counting the Number of Viable Cells:

Cells were stained with Hoechst 33342 (Dojindo, Kumamoto, Japan) and counted with ImageXpress (trade name) (Molecular Devices, California, USA).

### Scanning Electron Microscope (SEM):

NHDFs were plated using the same protocol as that of the sample. Recombinant OMD (0.01 μM, 0.1 μM, or 1 μM) was added to treatment groups. After 6 days of treatment, the NHDFs were fixed with a fixative solution, and the sample was subjected to SEM observation.

### Quantitative Real-Time RT-PCR:

RNA was extracted from cultured cells using RNeasy mini kit (Qiagen, Hilden, Germany), and RNA concentration was determined using a NanoDrop UV-Vis spectrophotometer (Thermo Fisher Scientific, Massachusetts, US). For qPCR, RNA was reverse-transcribed into first-strand cDNA using ReverTraAce qPCR RT Master Mix (Toyobo, Osaka, Japan). This cDNA was mixed with SYBR™ Green PCR Master Mix and primers of KRT1, KRT10, OCLN, CLDN1, FLG, LOR, IVL, KRT1, KRT10, COL1A1, p21, and OMD (Thermo Fisher Scientific, Massachusetts, US), and qPCR was performed using a real-time PCR instrument (Thermo Fisher Scientific) according to the manufacturer's protocol. Transcript levels were normalized to GAPDH.
Measurement of transepidermal water loss (TEWL): Vapometer SWL-5001 and SWL2040-09 probes (Delfin, Kupio, Finland) were used. For HSEs maintained in tissue culture, a medium adapted to environmental conditions of temperature and humidity for at least 15 minutes is used. TEWL was measured in three times for each HSE by the same inventor, and the average of each TEWL was calculated as a TEWL value.
Lactate dehydrogenase (LDH) assay: Viability/Cytotoxicity Multiplex Assay Kit (Dojindo, Kumamoto, Japan) was used, and assay was performed according to the instruction manual. For the HSE model, 100 μL of a medium was transferred to a 96-well plate and the LDH assay was performed. For cultured human fibroblasts and keratinocytes, a protein, such as OMD, was added, and the culture was allowed to stand for 48 hours, then 100 μL of the supernatant was transferred to a 96-well plate, and the LDH assay was performed.

### [Test Example 1] Cohort Analysis of Protein in Blood

In this test example, proteins in blood were clustered and visualized by a heatmap for each age to evaluate changes due to age. 3K SOMAscan proteomics z-score measurement values of 3283 plasma proteins from 3301 healthy individuals (1616 females and 1685 males) aged 18 to 76 years of the INTERVAL cohort (Cambridge, UK) were used. Changes in z-scores were visualized by clustering proteins with similar dynamics over human life span using R's pheatmap package (version 1.0.12). Plasma proteins with similar trajectories across females and males of all ages were grouped using unsupervised hierarchical clustering and clustering heatmap.

As a result, a unique cluster changing from a negative z-score to a positive z-score with increasing age was observed in females. The present inventors have found for the first time that this cluster is composed of eight proteins: osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).

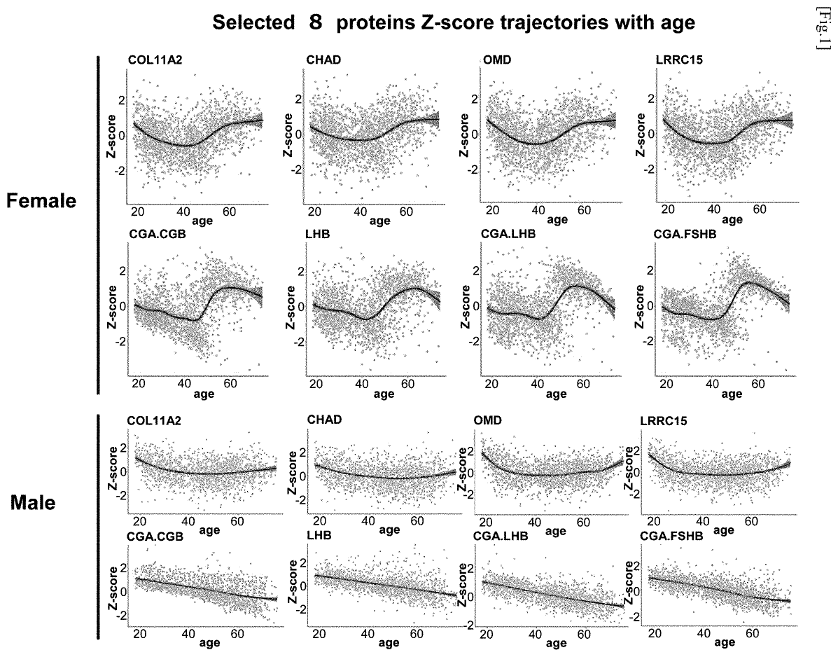

Results of analysis of details of changes in these eight proteins due to age is shown in FIG. 1. In females, these eight proteins decreased at about 20 to 40 years of age and increased at about 45 to 55 years of age. In males, the abundance of these proteins increased around 65 years of age and gradually decreased with age. Thus, these eight proteins were found to vary specifically in females.

Osteomodulin (OMD), chondroadherin (CHAD), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB) have a size of 66 kDa or less. Thus, the present inventors considered that these proteins can pass through blood vessels in the skin and reach the skin tissue, and thus affect the skin tissue and cells.

### [Test Example 2] Evaluation of Effects of Osteomodulin on Collagen Production

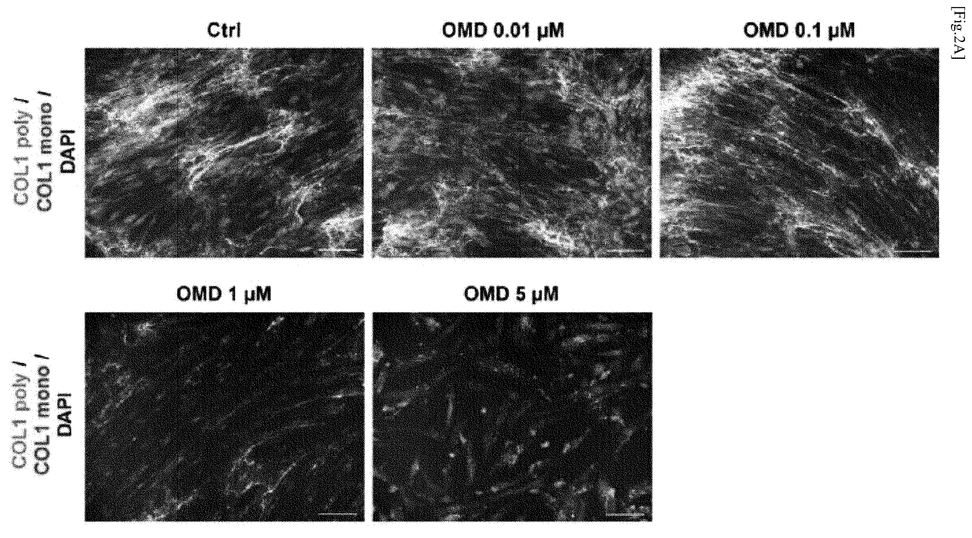

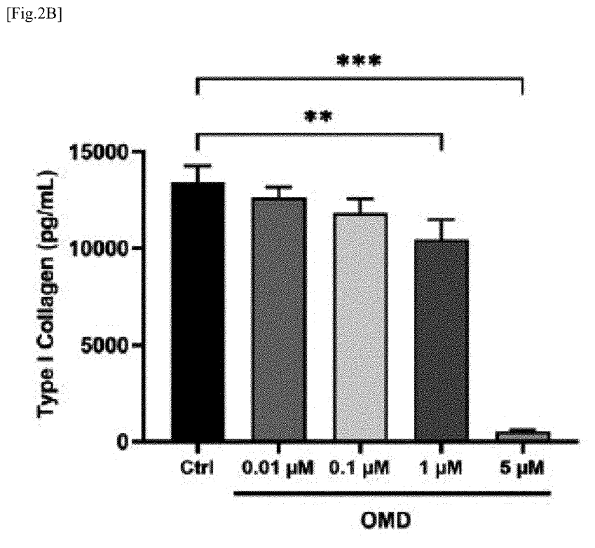

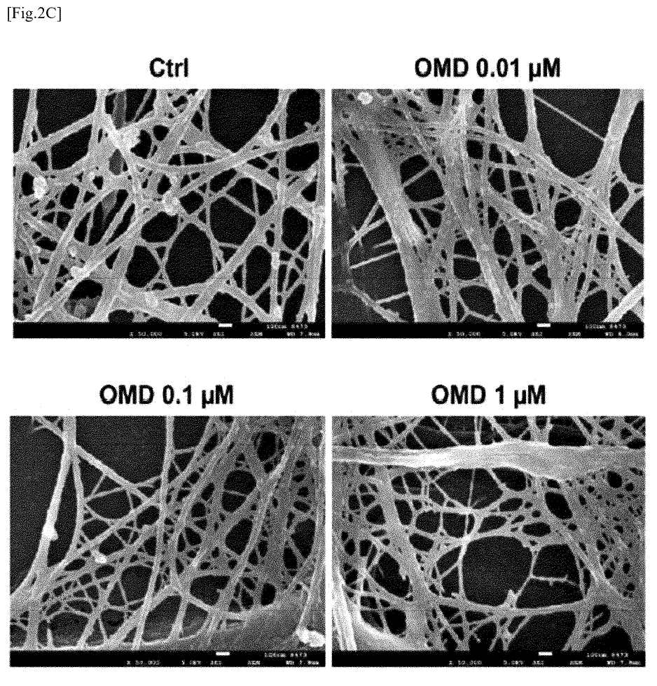

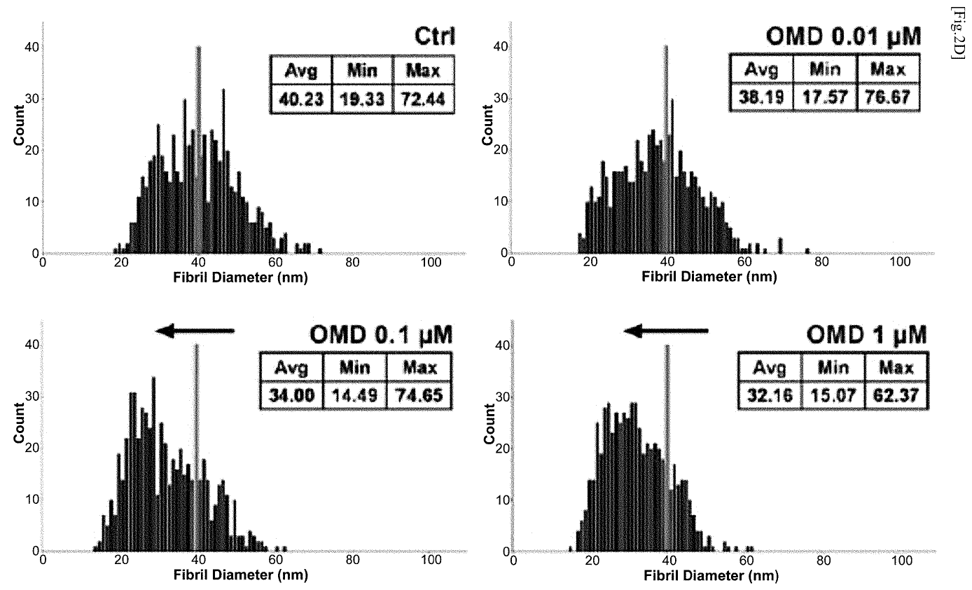

In this test example, effects of osteomodulin (OMD) on collagen production were evaluated. Specifically, OMD of each concentration was added to cultured normal human fibroblasts (NHDFs), the NHDFs were cultured for 6 days, and type I collagen was immunostained to obtain a fluorescence image (FIG. 2A). The type I collagen amount in each condition was measured by an enzyme-linked immunosorbent assay (ELISA) of collagen I αI (FIG. 2B). The concentrations of added OMD are shown in the graph. A sample to which OMD was not added was used as a control (Ctrl). Results of observation of the extracellular matrices of the NHDFs using a scanning electron microscope are shown in FIG. 2C. Furthermore, at least 600 fibrils were analyzed from scanning electron microscope images, and results of quantification of the collagen fiber diameters of the NHDFs and analysis of distribution of the collagen fiber diameters are shown in FIG. 2D.

As shown in FIG. 2A, type I collagen was stained in the extracellular compartment, and the staining intensity was low in the fibroblasts to which OMD was added. As shown in FIG. 2B, as a result of the ELISA, type I collagen production in the fibroblasts was inhibited depending on the concentration of OMD added. As shown in FIG. 2C, collagen fibers in the extracellular matrices were observed to be thinner when OMD was added compared with the control, finding that association of collagen is inhibited by OMD. As shown in FIG. 2D, in the NHDFs to which OMD was added, collagen fibers with a diameter smaller than 40 nm, which was the average diameter of collagen fibers in the control, increased (the bar at 40 nm of FIG. 2D is an auxiliary line for easier comparison with the control).

The present inventors have revealed that OMD inhibits the production of type I collagen in fibroblasts and reduces the diameter of collagen fibers.

### [Test Example 3] Evaluation of Effects of Osteomodulin on Expression of Genes Related to Skin Barrier Function

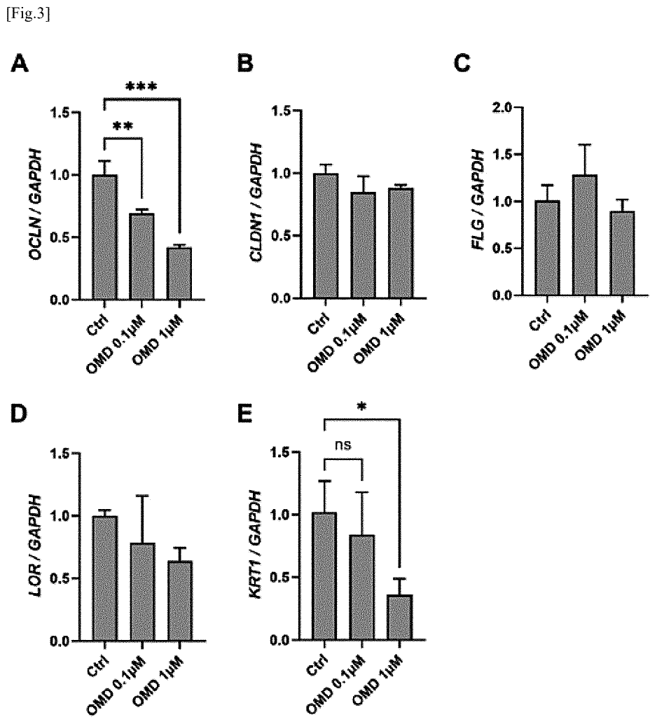

In this test example, effects of OMD on expression of genes related to skin barrier function were evaluated. Specifically, OMD was added to human keratinocytes (NHEKs) so as to be 0.1 μM or 1 μM, and gene expression of tight junction genes OCLN and CLDN1, terminal differentiation markers FLG and LOR, and an epidermal differentiation marker KRT1 was measured. GAPDH was used as a reference gene. A sample obtained by performing the test under the same conditions except for not adding OMD was used as a control. The results are shown in FIGS. 3A to 3E.

The addition of OMD to NHEKs reduced expression of the tight junction genes OCLN and CLDN1, the terminal differentiation marker LOR, and the epidermal differentiation marker KRT1 more than in the control (FIGS. 3A, 3B, 3D, and 3E). The expression of the terminal differentiation marker FLG was comparable to that of the control (FIG. 3C). Lactate dehydrogenase (LDH) release assay was performed for the purpose of confirming the presence or absence of cytotoxicity due to the addition of OMD. This found no difference in LDH release between the OMD-added group and the control. This result confirmed that cytotoxicity due to OMD did not affect the evaluation of each gene expression.

From the above, OMD was thought to inhibit the gene expression of OCLN, CLDN1, LOR, and KRT1 in NHEKs and to promote a decrease in the barrier function.

### [Test Example 4] Evaluation of Effects of Osteomodulin on Human Skin Equivalent (HSE) Model

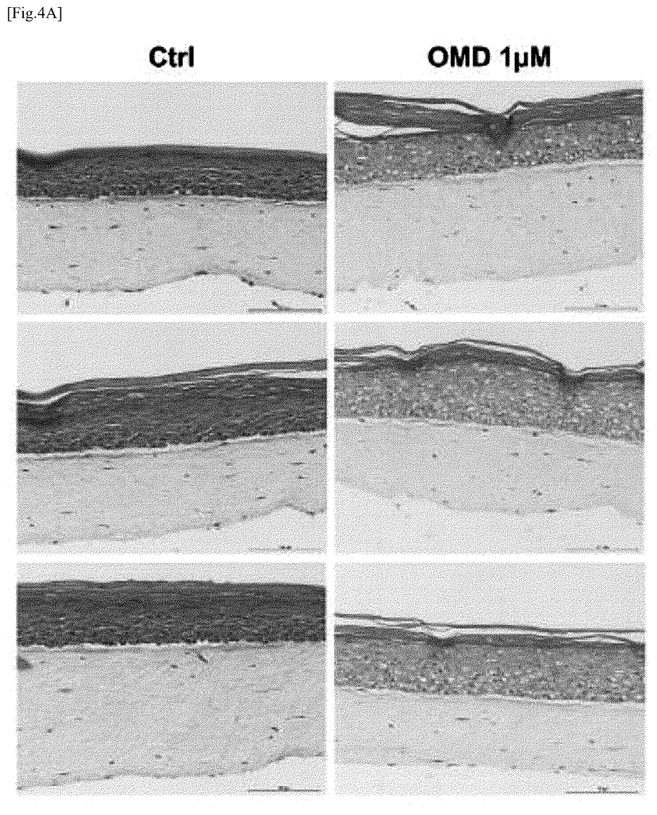

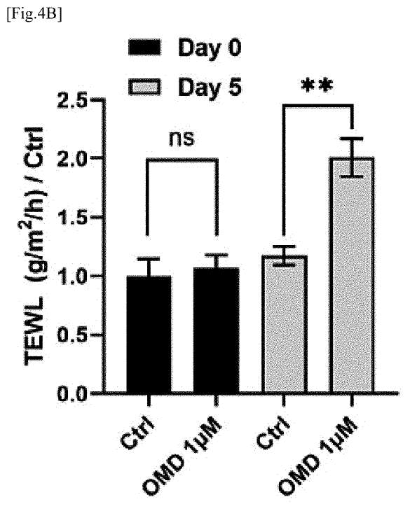

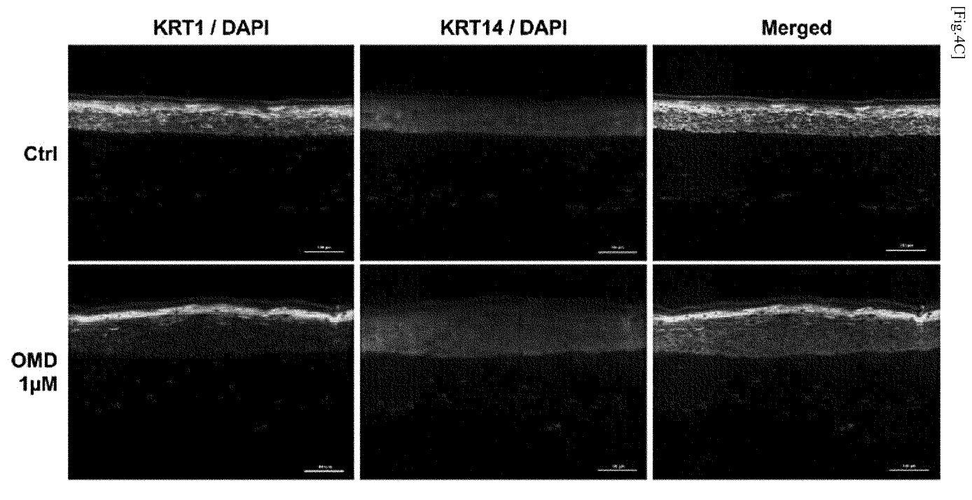

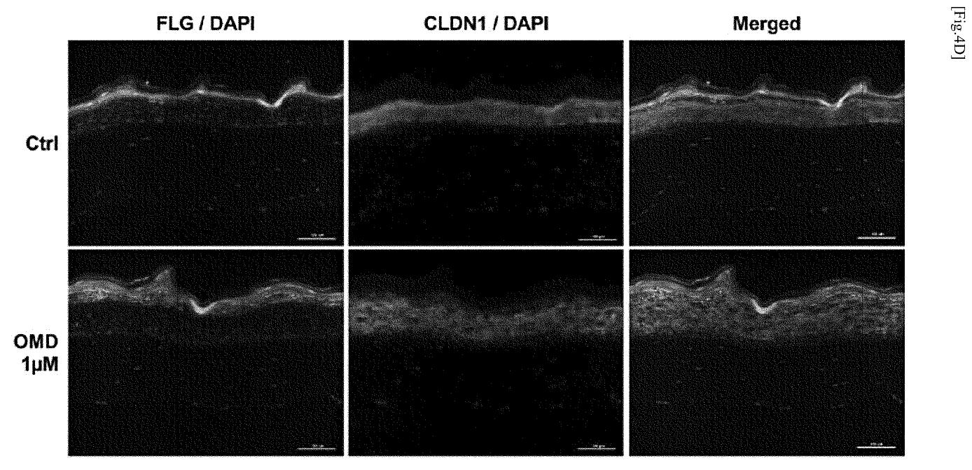

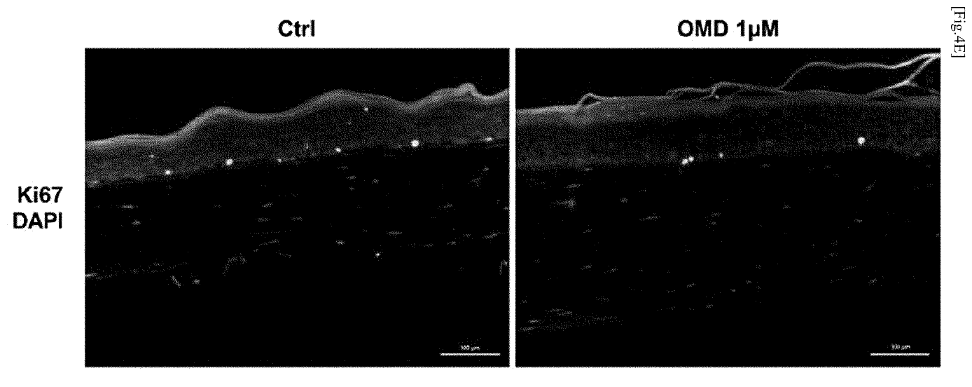

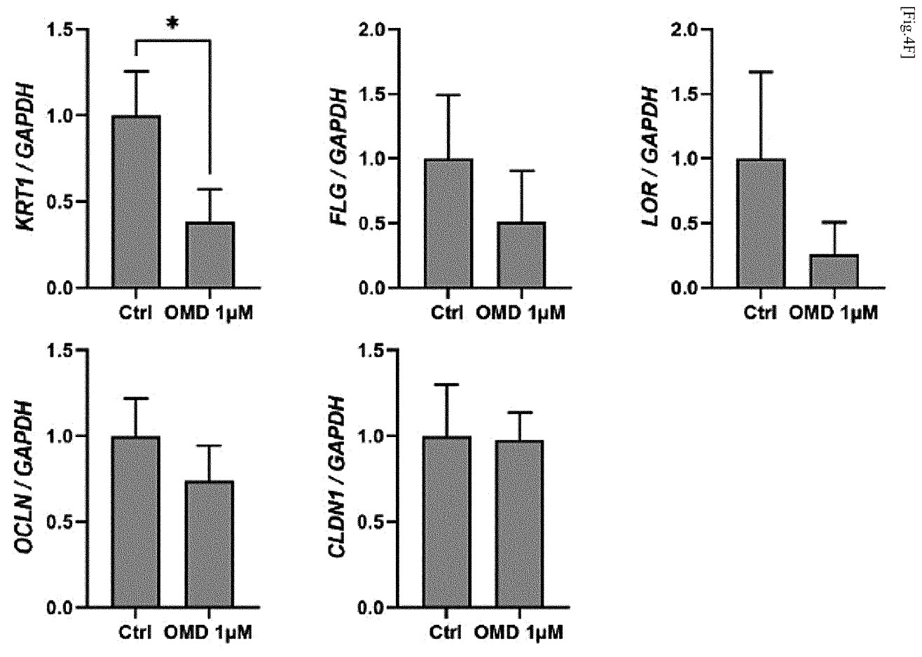

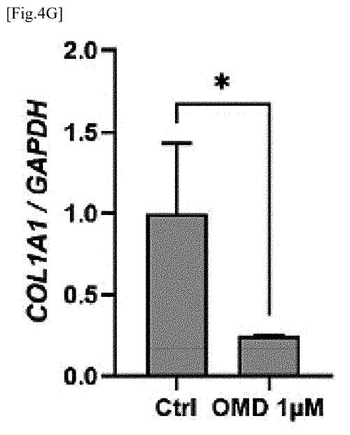

In this test example, a human skin equivalent (HSE) model (T-skinTM, EPISKIN SA) was cultured in a medium to which 1 μM OMD was added for 5 days, fixed with a 4% paraformaldehyde phosphate buffer (FUJIFILM Wako Pure Chemical Corporation, Osaka, Japan), embedded in paraffin, and H&E-stained and immunostained (FIG. 4A). In addition, transepidermal water loss (TEWL) was measured for the cells after cultured for 5 days (FIG. 4B).

For quantitative real-time polymerase chain reaction (RT-PCR), epidermis and dermis of the HSE were separated with forceps. Prior to RNA extraction, the tissue was crushed in Biomasher II (Nippi, Tokyo, Japan), centrifuged at 10000×g for 10 minutes, and the supernatant was used for extraction with RNeasy Mini Kit (Qiagen, Hilden, Germany).

NHDF were plated at a density of 38000 cells per well in a 24-well plate and cultured overnight in DMEM supplemented with 10% fetal bovine serum and 1% antibiotic-antimycotin. On the next day, the medium was replaced with DMEM (without fetal calf serum) supplemented with 1% antibiotic-antimycoplasma agent and 1 mg/mL L-ascorbic acid (013-12061, FUJIFILM Wako Pure Chemical Corporation, Osaka, Japan). OMD (0.01 μM, 0.1 μM, 1 μM, or 5 μM) was added to treatment groups. After 6 days of treatment, cells were washed with a PBS and incubated overnight at 4° C. with primary antibodies directed against collagen type I (C2456, monoclonal, Sigma-Aldrich, Missouri, USA, and NB600-408, polyclonal, Novus Biologicals, Colorado, USA). An Alexa488- or Alexa546-conjugated secondary antibody was then used, and the cells were incubated at 37° C. for 1 hour. HSE tissue sections were incubated at 37° C. for 2 hours with primary antibodies directed against Ki67 (ab16667, Abcam, Cambridge, UK), KRT1 (ab185628, Abcam), KRT14 (ab7800, Abcam), FLG (ab17808, Abcam), and CLDN1 (ab211737, Abcam). An Alexa488- or Alexa594-conjugated secondary antibody was then used, and the HSE tissue sections were incubated for 1 hour at 37° C. (Thermo Fisher Scientific, Waltham, MA, US). Samples were examined, and images were taken using an All-in-One Fluorescence Microscope BZ-X800 (Keyence, Osaka, Japan). A sample obtained under the same conditions as described above except for not adding OMD was used as a control (Ctrl).

As shown in FIG. 4A, in the HSE to which OMD was added, thickening of the epidermis was observed, and the number of vacuole-like structures observed around the cell nuclei increased. As shown in FIG. 4B, in the HSE to which OMD was added, TEWL increased, and the barrier function of the epidermis was inhibited. In addition, as shown in FIG. 4C, KRT1 distributed with a gradient in the control HSE, whereas in the HSE to which OMD was added, KRT1 was observed to be concentrated in the epidermis of the upper part of the basal portion; the addition of OMD caused abnormality in the KRT1 distribution in the HSE. This mechanism of action was thought to result in the inhibition of the epidermal barrier function by OMD. As shown in FIG. 4D, for the stained region by immunostaining of FLG/CLDN1, the stained regions of FLG and CLDN1 were clearly seen separately in the control HSE, but the stained regions were seen overlapping on the granular layer in the HSE to which OMD was added. As shown in FIG. 4E, in the HSE to which OMD was added, Ki67-positive cells decreased compared with the control, observing “inhibition of proliferation of cells in the basal layer”, which is one type of indicators indicating signs of intrinsic aging. As shown in FIG. 4F, in the HSE to which OMD was added, the gene expression of KRT1 involved in the inflammatory cytokine network was inhibited compared with the control, and the gene expression of terminal differentiation genes FLG and LOR and tight junction gene OCLN was also inhibited. Thus, exposure of the skin to OMD was found to inhibit the barrier function of the skin. FIG. 4G shows that the expression level of COL1A1 was reduced in the dermis in the HSE to which OMD was added.

The skin of menopausal females is known to change from a decrease in matrisome production to a dry state, and one of the causes is considered to be that OMD, which increases with advancing age, reduces the barrier function of the epidermis and inhibits collagen production and collagen fiber formation. From this, OMD was considered to be a useful biomarker for determining skin aging.

In addition, OMD changes the shape of dermal fibroblasts to an elongated shape, and this is considered to be due to loss of extracellular collagen and irregular signal transduction of cytoskeleton.

OMD affects the aggregate of collagen fibers and the diameter of collagen fibers. This leads to the consideration of the possibility that OMD may reduce the quality of type I collagen. In addition, the menopausal skin is known to have higher extensibility and laxity than the non-menopausal skin. OMD, which increases with advancing age, affects the diameter size of collagen fibers, and this was considered to be a possible cause for the higher extensibility and laxity of the menopausal skin.

Quality of collagen, such as orientation and fiber width, changes, in which intrinsic aging plays a more central role. Thus, OMD, which reduces the quality and quantity of collagen in the skin, is considered to be particularly useful as a biomarker for determining skin aging.

Impairing the production quantity and quality of type I collagen causes wrinkles, sagging, and the like, which adversely affects the appearance of the skin. In addition, in aged skin and menopausal skin, skin dryness causes discomfort and reduces the barrier function, and thus is a major concern. OMD, which increases with advancing age, affects both the epidermis and dermis of the skin and thus is said to be a useful biomarker.

OMD is a useful biomarker for determining skin aging. Thus, examining a change in the expression level or concentration of OMD in cells or tissues as a target is considered to be able to screen an OMD-inhibiting component, an OMD-production-suppressing component, and a component effective for inhibiting aging of the dermis and epidermis.

### [Test Example 5] Evaluation of Effects of CHAD and LRRC15 on Collagen Production

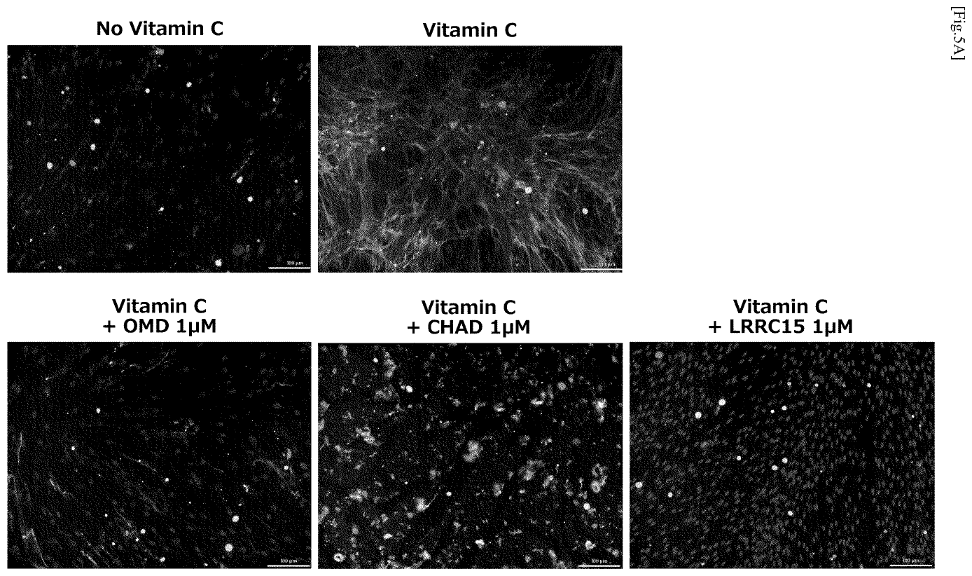

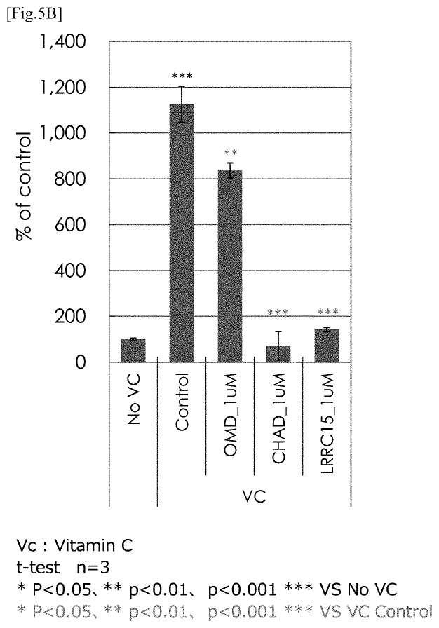

In this test example, effects of chondroadherin (hereinafter also referred to as CHAD) and leucine-rich repeat-containing protein 15 (hereinafter also referred to as LRRC15) on collagen production were evaluated. Specifically, 1 mg/mL L-ascorbic acid (Vitamin C) (013-12061, FUJIFILM Wako Pure Chemical Corporation, Osaka, Japan) and 1 μM of OMD, CHAD, or LRRC15 were added to cultured normal human fibroblasts (NHDFs), the NHDFs were cultured for 6 days, and type I collagen was immunostained to obtain a fluorescence image (FIG. 5A). The type I collagen amount in the culture supernatant in each condition was measured by an enzyme-linked immunosorbent assay (ELISA) of collagen I α1. The measured value of each sample was calculated based on the measured value of a negative control taken as 100 (FIG. 5B). A sample obtained by adding only L-ascorbic acid without adding CHAD and LRRC15 was used as a control. A sample to which L-ascorbic acid and proteins (CHAD and LRRC15) were not added was used as the negative control.

As shown in FIG. 5A, the control to which L-ascorbic acid was added showed high staining intensity, but in the fibroblasts to which 1 μM of OMD, CHAD, or LRRC15 was added in addition to L-ascorbic acid, the staining intensity significantly decreased. As shown in FIG. 5B, as a result of the ELISA, the production of type I collagen was inhibited in the fibroblasts to which 1 μM of OMD, CHAD, or LRRC15 was added. In particular, the effect of inhibiting type I collagen production was significant in the fibroblasts to which CHAD and LRRC15 were added.

The present inventors have revealed that CHAD and LRRC15 inhibit the production of type I collagen and disturb the morphology and secretion state of collagen fibers. Similarly to OMD, CHAD or LRRC15, which is a protein of 66 kDa or less, can pass through blood vessels in the skin and reach the skin tissue and thus is considered to have a more significant effect on the tissues and cells in the skin when the blood concentration increases with advancing age and/or the like.

### INDUSTRIAL APPLICABILITY

The present invention can be used in all technical fields requiring a biomarker for determining skin aging and its use, detection of the progression state of skin aging, determination of skin aging, and screening of a substance for improving skin aging, for example, detection of symptoms due to skin aging, prediction of degree of progression of symptoms, prediction of risk of progression of symptoms, investigation of the cause of skin aging, confirmation of the effect of inhibiting skin aging by a drug and/or the like, confirmation of the effect of treating skin aging, and the like, and can be particularly utilized in the fields of pharmaceuticals, cosmetics, foods, and the like.

1.-4. (canceled)

5. A method for determining skin aging in a subject, the method comprising quantifying at least one protein or a fragment thereof in a blood sample from the subject, the protein being selected from the group consisting of osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).

6. A method for screening a substance for improving skin aging, the method comprising examining a change in expression level or concentration of at least one protein or a fragment thereof in a blood sample from a subject before and after ingestion of a test substance by the subject, the protein being selected from the group consisting of osteomodulin (OMD), chondroadherin (CHAD), collagen type XI alpha 2 chain (COL11A2), leucine-rich repeat-containing protein 15 (LRRC15), Glycoprotein hormones alpha chain/Choriogonadotropin subunit beta 3/Choriogonadotropin subunit beta 7 (CGA/CGB3/CGB7), lutropin subunit beta (LHB), glycoprotein hormones alpha chain/lutropin subunit beta (CGA/LHB), and glycoprotein hormones alpha chain/follitropin subunit beta (CGA/FSHB).
